(19) United States
(12) Patent Application Publication
(10) Pub. No.: US 2022/0071567 A1
(43) Pub. Date: Mar. 10, 2022

(54) INFORMATION PROCESSING APPARATUS, INFORMATION PROCESSING METHOD AND NON-TRANSITORY STORAGE MEDIUM

(71) Applicant: TOYOTA JIDOSHA KABUSHIKI KAISHA, Toyota-shi Aichi-ken (JP)

(72) Inventors: Takaharu Ueno, Nagoya-shi (JP); Yu Nagata, Chofu-shi (JP); Yurika Tanaka, Yokosuka-shi (JP); Ryosuke Kobayashi, Nagakute-shi (JP); Shintaro Matsutani, Kariya-shi (JP); Syouta Komatsu, Toyota-shi (JP)

(21) Appl. No.: 17/465,031

(22) Filed: Sep. 2, 2021

(30) Foreign Application Priority Data
Sep. 4, 2020 (JP) 2020-148667

(51) Int. Cl. A61B 5/00; G16H 20/10; G16H 80/00; G16H 10/60

(52) U.S. Cl. CPC A61B 5/7275; A61B 5/7282; G16H 10/60; G16H 80/00; G16H 20/10

(57) ABSTRACT

A controller comprising at least one processor of an information processing apparatus executes: acquiring first information about a condition of a body of a user; and acquiring second information about a condition of an environment around the user. Then, the controller of the information processing apparatus executes: executing a predetermined judgment process for judging whether predetermined symptoms occur on the user within a predetermined period, based on the first information and the second information; and notifying the user that predetermined medicine for coping with the predetermined symptoms is to be used, if a positive judgment is made in the predetermined judgment process.

### CROSS REFERENCE TO THE RELATED APPLICATION

This application claims the benefit of Japanese Patent Application No. 2020-148667, filed on Sep. 4, 2020, which is hereby incorporated by reference herein in its entirety.

### BACKGROUND

### Technical Field

The present disclosure relates to an information processing apparatus, an information processing method and a non-transitory storage medium.

### Description of the Related Art

Japanese Patent Application No. 2003-173375 discloses a life management system. The life management system in Japanese Patent Application No. 2003-173375 is provided with a life management terminal apparatus that detects that a user is having a meal based on information measured from the user. The life management system is provided with means for, when it is detected by the life management terminal apparatus that the user has started or ended a meal, inquiring the user of content of the meal after a predetermined period after the detection, and means for acquiring an answer to the inquiry from the user. The life management system is further provided with presentation means for, when the life management terminal apparatus detects that the user has ended a meal or after a predetermined time after it is detected that the user has started a meal, presenting information about medicine that the user should take after the meal to the user and prompting the user to take the medicine.

### SUMMARY

An object of the present disclosure is to prompt a user to use medicine irregularly used, at an appropriate timing.

An information processing apparatus according to a first aspect of the present disclosure includes a controller comprising at least one processor configured to execute:

acquiring first information about a condition of a body of a user;

acquiring second information about a condition of an environment around the user;

executing a predetermined judgment process for judging whether predetermined symptoms occur on the user within a predetermined period, based on the first information and the second information; and

notifying the user that predetermined medicine for coping with the predetermined symptoms is to be used, if a positive judgment is made in the predetermined judgment process.

An information processing method according to a second aspect of the present disclosure includes:

acquiring first information about a condition of a body of a user;

acquiring second information about a condition of an environment around the user;

executing a predetermined judgment process for judging whether predetermined symptoms occur on the user within a predetermined period, based on the first information and the second information; and

notifying the user that predetermined medicine for coping with the predetermined symptom is to be used, if a positive judgment is made in the predetermined judgment process.

A non-transitory storage medium according to a third aspect of the present disclosure is a non-transitory storage medium storing a program causing a computer to execute an information processing method, the information processing method including:

acquiring first information about a condition of a body of a user;

acquiring second information about a condition of an environment around the user;

executing a predetermined judgment process for judging whether predetermined symptoms occur on the user within a predetermined period, based on the first information and the second information; and

notifying the user that predetermined medicine for coping with the predetermined symptoms is to be used, if a positive judgment is made in the predetermined judgment process.

According to the present disclosure, it is possible to prompt a user to use medicine irregularly used, at an appropriate timing.

### BRIEF DESCRIPTION OF THE DRAWINGS

FIG. 1 is a diagram illustrating a schematic configuration of a management system according to a first embodiment.

FIG. 2 is a block diagram schematically illustrating an example of a functional configuration of each of a user terminal and a management server.

FIG. 3 is a diagram illustrating an example of a table configuration of history information in the first embodiment.

FIG. 4 is a flow chart of a notification process.

FIG. 5 is a sequence diagram illustrating information and process flows in the management system.

FIG. 6 is a diagram illustrating a schematic configuration of a management system in a second embodiment.

FIG. 7 is a diagram illustrating an example of a table configuration of history information in the second embodiment.

### DESCRIPTION OF THE EMBODIMENTS

An information processing apparatus according to a first aspect of the present disclosure is an information processing apparatus that performs management of use of medicine by a user. Here, the user is a person who needs to use predetermined medicine to cope with predetermined symptoms that irregularly appear on his body. In other words, the user irregularly uses the predetermined medicine.

A controller comprising at least one processor in the information processing apparatus according to the first aspect of the present disclosure acquires first information about the condition of a body of a user. Here, the first information is, for example, information about the body temperature, heart rate, blood pressure, presence/absence of coughs, presence/absence of sneezes or the like about the user. Further, the controller in the information processing apparatus acquires second information about the condition of an environment around the user. Here, the second information is, for example, information including meteorological information such as atmospheric temperature, atmospheric pressure, humidity and weather. Further, the second information is, for example, information including information about an amount of house dust scattered, an amount of pollen scattered or the like around the user.

When predetermined symptoms occur on the body of the user, a sign of the occurrence of the predetermined symptoms may appear in the condition of the user's body. At this time, the environment around the user influences the condition of the body of the user. Therefore, the controller executes a predetermined judgment process based on the first information and the second information. The predetermined judgment process is a process for judging whether the predetermined symptoms will occur on the user within a predetermined period or not. Then, if a positive judgment is made in the predetermined judgment process, the controller notifies the user that the user should use the predetermined medicine.

By being notified that the user should use the predetermined medicine as described above, the user can grasp that he should use the predetermined medicine. Thus, it is possible to prompt the user to use the medicine that is irregularly used, at an appropriate timing by the information processing apparatus.

Specific embodiments of the present disclosure will be described below based on drawings. The dimensions, materials, shapes, relative arrangement and the like of components described in the present embodiments are not intended to limit the technical scope of the present disclosure thereto unless otherwise stated.

### First Embodiment

### (Outline of System)

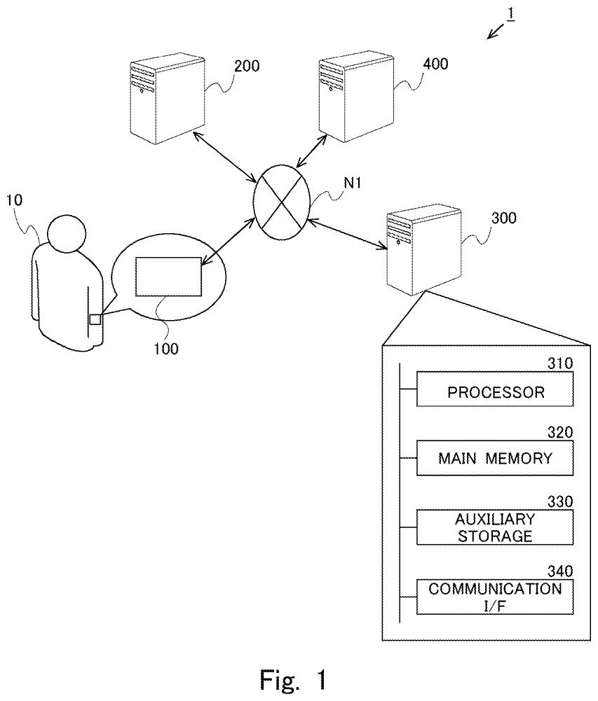

A management system 1 in the present embodiment will be described based on FIG. 1. FIG. 1 is a diagram illustrating a schematic configuration of a management system 1 according to the present embodiment. The management system 1 is configured including a user terminal 100, an environment server 200, a management server 300 and a medical server 400.

In the management system 1, the user terminal 100, the environment server 200, the management server 300 and the medical server 400 are mutually connected via a network N1. As the network N1, for example, a WAN (wide area network), which is a worldwide public communication network such as the Internet, or a telephone communication network for mobile phones or the like may be adopted.

(User Terminal)

The user terminal 100 is a terminal related to a user 10. The user terminal 100 is, for example, a wearable terminal that the user 10 uses. Here, the user 10 is a person who needs to use predetermined medicine to cope with predetermined symptoms that irregularly appear on his body. For example, if the user 10 is an asthmatic patient, coughs, palpitations or the like can be shown as examples of the predetermined symptoms. When the predetermined symptoms occur, the user 10 uses predetermined medicine for the predetermined symptoms (which may be hereinafter referred to as “predetermined medicine”). Here, as a method of using the predetermined medicine, for example, taking (taking only when necessary), aspiration, application, injection or the like of the predetermined medicine can be given. Since the predetermined symptoms irregularly occur on the body of the user 10, the user 10 needs to irregularly use the predetermined medicine. For example, if the user 10 is an asthmatic patient, the user 10 needs to aspire aspiration medicine, which is predetermined medicine for controlling symptoms of asthma, when the symptoms due to asthma occurs.

(Environment Server)

The environment server 200 is a server that collects information about conditions of environments of regions. Here, the condition of each environment (environmental condition) collected by the environment server 200 includes, for example, atmospheric temperature, atmospheric pressure, humidity and weather.

(Management Server)

The management server 300 is a server that performs management of use of the predetermined medicine by the user 10. It is assumed that the environmental condition of a region where the user 10 exists influences the condition of the body of the user 10. Therefore, the management server 300 receives information about the environment of the region where the user 10 exists (which may be hereinafter referred to as “environmental information”) from the environment server 200 via the network N1. Further, the management server 300 transmits notification information for notifying the user 10 that the predetermined medicine should be used, to the user terminal 100 via the network N1. Note that details of a timing of transmitting the notification information from the management server 300 to the user terminal 100 will be described later.

The management server 300 is configured including a computer provided with a processor 310, a main memory 320, an auxiliary storage 330 and a communication interface (communication I/F) 340. The processor 310 is, for example, a CPU (central processing unit) or a DSP (digital signal processor). The main memory 320 is, for example, RAM (random access memory). The auxiliary storage 330 is, for example, ROM (read-only memory). The auxiliary storage 330 is, for example, an HDD (hard disk drive) or a disk recording medium such as a CD-ROM, a DVD disc or a Blu-ray Disc. Further, the auxiliary storage 330 may be a removable medium (portable storage medium). Here, as the removable medium, a USB memory or an SD card is shown as an example. The communication I/F 340 is, for example, a LAN (local area network) interface board or a wireless communication circuit for wireless communication.

In the management server 300, an operating system (OS), various kinds of programs, various kinds of information tables and the like are stored in the auxiliary storage 330. Further, in the management server 300, by the processor 310 loading a program stored in the auxiliary storage 330 to the main memory 320 and executing the program, various kinds of functions as described later can be realized. However, a part or all of the functions of the management server 300 may be realized by a hardware circuit like an ASIC or an FPGA. Note that the management server 300 is not necessarily required to be realized by a single physical configuration but may be configured with a plurality of computers that cooperate with one another. Further, the environment server 200 is configured including a computer, similarly to the management server 300.

(Medical Server)

The medical server 400 is a server apparatus in which the health condition of the user 10 is recorded. The medical server 400 is a server related to a predetermined medical institution. Here, the medical server 400 is a server apparatus in which a medical book about the user 10 is recorded. Further, the predetermined medical institution is a medical institution where the user 10 sees a doctor to cope with the predetermined symptoms. Note that the medical server 400 is configured including a computer, similarly to the management server 300.

(Functional Configuration)

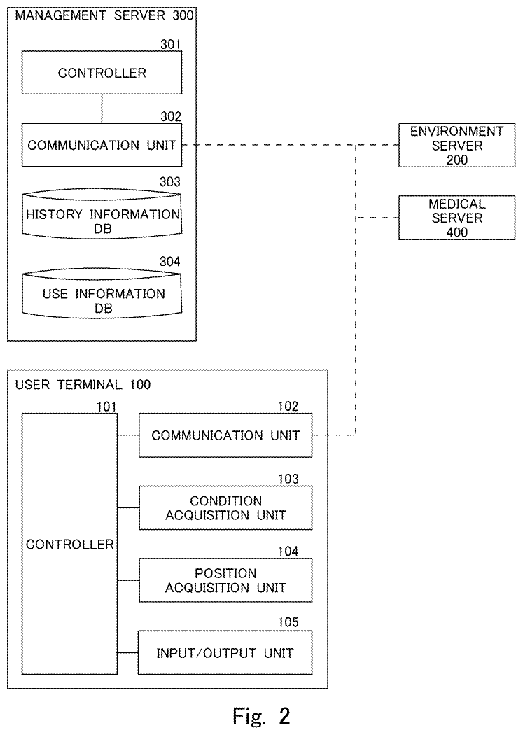

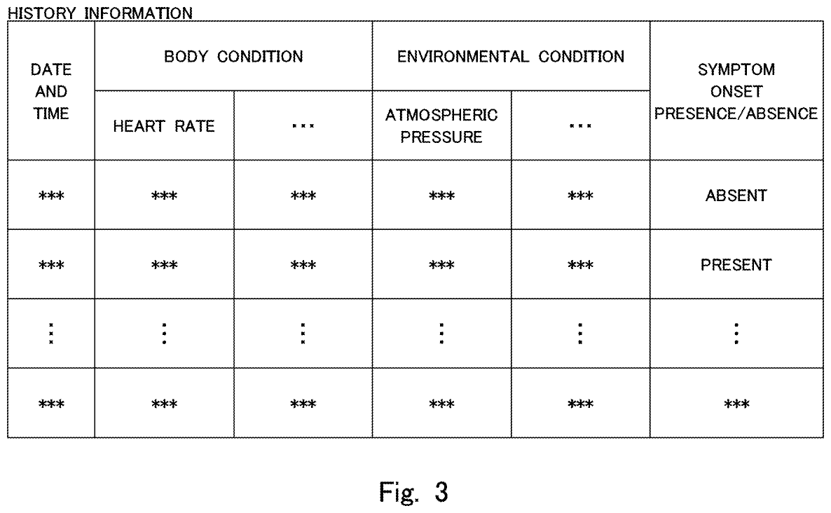

Next, a functional configuration of each of the user terminal 100, the environment server 200 and the management server 300 constituting the management system 1 according to the present embodiment will be described based on FIGS. 2 and 3. FIG. 2 is a block diagram schematically illustrating an example of the functional configuration of each of the user terminal 100 and the management server 300.

(User Terminal)

The user terminal 100 is configured including a controller 101, a communication unit 102, a condition acquisition unit 103, a position acquisition unit 104 and an input/output unit 105. The controller 101 has a function of performing arithmetic processing for controlling the user terminal 100. The controller 101 can be realized by a processor of the user terminal 100. The communication unit 102 has a function of connecting the user terminal 100 to the network N1. The communication unit 102 can be realized by a communication I/F of the user terminal 100.

The condition acquisition unit 103 has a function of acquiring information about the condition of the body (the body condition) of the user 10. Here, for example, the body temperature, heart rate, blood pressure and presence/absence of coughs about the user 10 are acquired by the condition acquisition unit 103 as the information about the body condition of the user 10. The condition acquisition unit 103 can be realized by a biosensor of the user terminal 100. The controller 101 transmits the information about the body condition (which may be hereinafter referred to as “condition information”) about the user 10 to the management server 300 via the communication unit 102.

The position acquisition unit 104 has a function of acquiring a current position of the user 10. The position acquisition unit 104 can be realized by a GPS receiver of the user terminal 100. The controller 101 transmits position information, which is information about the current position of the user 10 acquired by the position acquisition unit 104, to the management server 300.

The input/output unit 105 has a function for the user 10 to input various pieces of information to the user terminal 100. Further, the input/output unit 105 has a function of displaying various pieces of information to the user 10. The input/output unit 105 can be realized by a touch panel of the user terminal 100. The controller 101 receives notification information transmitted from the management server 300, by the communication unit 102. When receiving the notification information, the controller 101 causes the input/output unit 105 to display that the predetermined medicine should be used, based on the notification information. When having used the predetermined medicine, the user 10 inputs information indicating that the user 10 has used the predetermined medicine to the input/output unit 105. The controller 101 generates use information based on the input information. Here, the use information includes information about a date and time when the user 10 used the predetermined medicine. Then, the controller 101 transmits the use information to the management server 300 via the communication unit 102.

(Management Server)

The management server 300 is configured including a controller 301, a communication unit 302, a history information database (a history information DB) 303 and a use information database (a use information DB) 304. The controller 301 has a function of performing arithmetic processing for controlling the management server 300. The controller 301 can be realized by the processor 310 of the management server 300. The communication unit 302 has a function of connecting the management server 300 to the network N1. The communication unit 302 can be realized by the communication I/F 340 of the management server 300.

The controller 301 receives the use information from the user terminal 100 by the communication unit 302. The controller 301 stores the received use information into the use information DB 304. The use information DB 304 can be realized by the auxiliary storage 330 of the management server 300. The management server 300 can grasp the time when the user 10 used the predetermined medicine based on the use information stored in the use information DB 304.

In the history information DB 303, the body condition of the user 10 and the environmental condition of a region where the user 10 existed during a certain period in the past are stored, being associated with each other. Here, the certain period in the past (which may be hereinafter referred to simply as a “past period”) is a period before the user 10 used the predetermined medicine. For example, the controller 301 receives information about a body condition of the user 10 in the past period from the user terminal 100. Further, the controller 301 receives information about an environmental condition of a region where the user 10 existed in the past period from the environment server 200. Then, the controller 301 stores these pieces of information into the history information DB 303. In other words, the information about the body condition about the user 10 stored in the history information DB 303 shows a history of the body condition of the user 10 when the user 10 does not use the predetermined medicine. Note that the history information DB 303 can be realized by the auxiliary storage 330 of the management server 300.

FIG. 3 is a diagram illustrating an example of a table configuration of history information in the present embodiment. As illustrated in FIG. 3, the history information includes date and time fields, body condition fields, environmental condition fields and symptom onset presence/absence fields. In each date and time field, a date and time in the past period is input. In each body condition field, the body condition of the user 10 at a date and time input in a corresponding date and time field is input. Specifically, the body temperature, heart rate, blood pressure, presence/absence of coughs and the like about the user 10 are input in the body condition field. In each environmental condition field, an environmental condition of a region where the user 10 existed at a date and time input in a corresponding date and time field is input. Specifically, in the environmental condition field, atmospheric temperature, atmospheric pressure, humidity, weather and the like of the region where the user 10 existed are input. In each symptom onset presence/absence field, information about whether the predetermined symptoms occurred on the user 10 or not at a date and time input in a corresponding date and time field is input. Specifically, if the predetermined symptoms occurred on the user 10, “present” is input in the symptom onset presence/absence field. If the predetermined symptoms did not occur on the user 10, “absent” is input in the symptom onset presence/absence field. Here, the controller 301 judges whether the predetermined symptoms occurred on the user 10 or not, based on the body condition of the user 10 input in the body condition field. Here, the controller 301 judges whether the predetermined symptoms occurred on the user 10 or not, for example, based on information about presence/absence of coughs input in the body condition field. Note that, as a method for judging whether the predetermined symptoms occurred on the user 10 or not, a publicly known method can be adopted.

The controller 301 acquires condition information by receiving the condition information from the user terminal 100 by the communication unit 102. Further, the controller 301 receives position information by the communication unit 302. The controller 301 identifies a region where the user 10 exists based on the received position information. The controller 301 acquires environmental information about the region where the user 10 exists. Then, the controller 301 executes a predetermined judgment process based on the acquired condition information, the acquired environmental information and the history information stored in the history information DB 303. Here, the predetermined judgment process is a process for judging whether the predetermined symptoms will occur on the user 10 within a predetermined period or not. Specifically, the controller 301 judges whether a predetermined condition is satisfied or not based on the condition information, the environmental information and the history information in the predetermined judgment process. Here, the predetermined condition is a condition for it being assumed that the predetermined symptoms occur on the user 10 within the predetermined period. The predetermined condition is, specifically, that a body condition in the condition information and an environmental condition in the environmental information are similar to a body condition and an environmental condition before occurrence of the predetermined symptoms on the user 10 in the history information. If the predetermined condition is satisfied, it is assumed that the predetermined symptoms will occur on the user 10 within the predetermined period. Therefore, if the predetermined condition is satisfied, the controller 301 judges that the predetermined symptoms will occur on the user 10 within the predetermined period. Note that the condition information in the present embodiment corresponds to “first information” according to the present disclosure. The environmental information in the present embodiment corresponds to “second information” according to the present disclosure.

Further, if a positive judgment is made in the predetermined judgment process, the controller 301 estimates symptom onset time when the predetermined symptoms occur on the user 10. Specifically, the controller 301 acquires a date and time in the history information when the body condition in the condition information and the environmental condition in the environmental information are similar to a body condition and an environmental condition before occurrence of the predetermined symptoms on the user 10 in the history information. Then, the controller 301 calculates a difference between the acquired date and time and a date and time in the history information when the predetermined symptoms occurred on the user 10 in the past, which is closest to the acquired date and time. Then, the controller 301 estimates symptom onset time of the user 10 based on the calculated difference between the dates and times.

Further, the controller 301 identifies use time for when the user 10 should use the predetermined medicine, based on the estimated symptom onset time. The controller 301 identifies, for example, time when the effect of use of the predetermined medicine appears before the symptom onset time as the use time. Further, at this time, the controller 301 identifies the use time based on time when the user 10 used the predetermined medicine last in the use information stored in the use information DB 304. The controller 301 identifies time when a predetermined interval has elapsed after the user 10 used the predetermined medicine as the use time. Here, the predetermined interval is an interval at which the predetermined medicine can be used again after being used once. By deciding the use period as described above, it is possible to prevent the user 10 from deciding the use time at an interval shorter than the predetermined interval after using the predetermined medicine.

The controller 301 transmits notification information including the use time to the user terminal 100 via the communication unit 302. The user terminal 100 receiving the notification information displays that the predetermined medicine should be used, together with the use time, based on the notification information. Thereby, the user 10 can grasp that the user 10 should use the predetermined medicine at the use time.

(Notification Process)

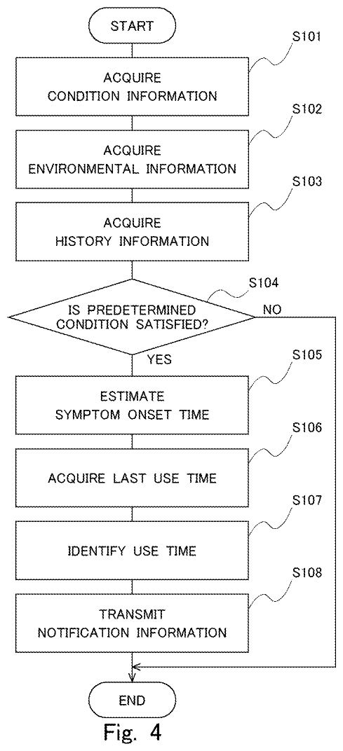

A description will be made on a notification process executed by the controller 301 of the management server 300 in the management system 1 according to the present embodiment, based on FIG. 4. FIG. 4 is a diagram illustrating a flowchart of the notification process. The notification process is a process for the management server 300 to transmit notification information to the user terminal 100.

In the notification process, first, condition information is acquired by receiving the condition information from the user terminal 100 in S101. In S102, environmental information is acquired by receiving the environmental information from the environment server 200. In S103, history information stored in the history information DB 303 is acquired. Next, in S104, it is judged whether the condition information, the environmental information and the history information satisfy the predetermined condition or not. In other words, the predetermined judgment process is executed in S104. If a negative judgment is made in S104, the notification process is ended. If a positive judgment is made in S104, symptom onset time of the user 10 is estimated in S105. Next, in S106, time when the user 10 used the predetermined medicine last (the last use time) is acquired from the use information DB 304. Next, in S107, time to use the predetermined medicine is identified. Next, in S108, notification information including the use time is transmitted to the user terminal 100. Then, the notification process is ended.

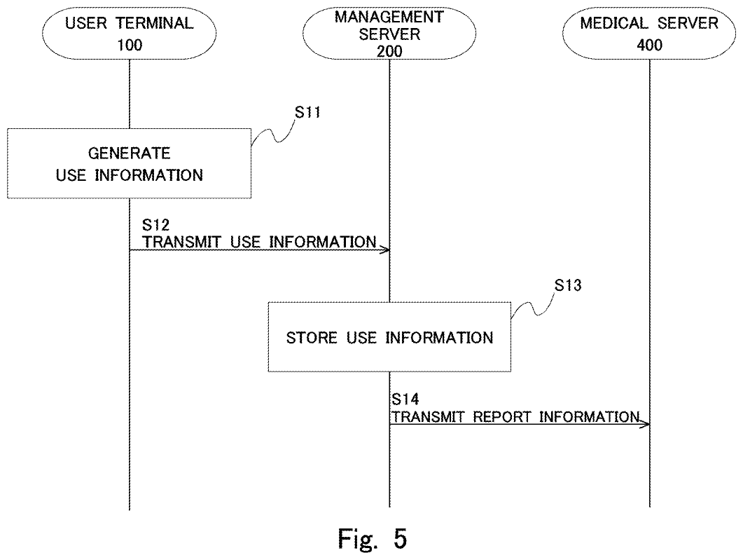

Next, a description will be made on information and process flows among the user terminal 100, the management server 300 and the medical server 400 in the management system 1, based on FIG. 5. FIG. 5 is a sequence diagram illustrating the information and process flows in the management system 1.

When the user 10 inputs information showing that the user 10 has used the predetermined medicine, to the user terminal 100, the user terminal 100 generates use information (S11). Next, the user terminal 100 transmits the use information to the management server 300 (S12). Then, the management server 300 stores the received use information into the use information DB 304 of the management server 300 (S13). Next, the management server 300 transmits report information to the medical server 400 (S14). Here, the report information is information that includes body conditions of the user 10 and environmental conditions of regions where the user 10 existed in periods before and after a time point when the user 10 used the predetermined medicine (which may be hereinafter referred to as “particular periods”). Here, the particular periods are periods set in advance.

By the medical server 400 receiving the report information, a doctor in charge of the user 10 in the predetermined medical institution can grasp the body conditions of the user 10 and the environmental conditions of the regions where the user 10 exists in the particular periods. In other words, the doctor in charge of the user 10 can grasp the body conditions and the environmental conditions before and after the user 10 used the predetermined medicine. Thereby, the doctor in charge of the user 10 can consider a treatment method or a coping method for the predetermined symptoms that occur on the user 10.

As described above, by the notification information being transmitted to the user terminal 100 in the management system 1, the user 10 can grasp that the user 10 should use the predetermined medicine. Thus, it is possible to prompt the user 10 to use the predetermined medicine that is irregularly used by the user 10, at an appropriate timing by the management system 1.

(Modification)

Note that, in the present embodiment, the management server 300 executes the predetermined judgment process based on the condition information, the environmental information and the history information. However, the management server 300 may perform the predetermined judgment process using information about change in an environmental condition forecast for a region where the user 10 exists (which may be hereinafter referred to simply as “forecast information”) in addition to the environmental information. In other words, the management server 300 executes the predetermined judgment process based on the condition information, the environmental information, the forecast information and the history information. Here, the predetermined judgment process is a process for judging whether a predetermined condition is satisfied or not based on the condition information, the environmental information, the forecast information and the history information. The predetermined condition is that a body condition in the condition information and change between a current environmental condition and a forecast environmental condition in the environmental information and the forecast information are similar to a body condition and transition of change in environmental information in the history information. In other words, the management server 300 executes the predetermined judgment process based on the body condition in the condition information and the body condition in the history information, the change between the current environmental condition and the forecast environmental condition in the environmental information and the forecast information, and the transition in the change in the environmental condition in the history information.

The management server 300 may execute the predetermined judgment process not depending on the history information but based on the condition information and the environmental information. In other words, the management server 300 judges whether the condition information and the environmental information satisfy a predetermined condition or not. The predetermined condition in this case is, for example, that the body condition of the user 10 and the environmental condition of a region where the user 10 exists show predetermined values.

In the present embodiment, the environmental information is information about the atmospheric temperature, atmospheric pressure, humidity and weather of a region where the user 10 exists. The environmental information, however, is not necessarily limited to the information about the atmospheric temperature, atmospheric pressure, humidity and weather of the region where the user 10 exists. The environmental information may include, for example, information about an amount of house dust scattered around the user 10. In this case, the amount of house dust scattered is acquired from an air purifier existing around the user 10. Further, the predetermined symptoms are not necessarily limited to symptoms of asthma. A case where the predetermined symptoms that occur on the user 10 are sneezes due to pollen allergy is assumed. In this case, the environmental information may include, for example, information about an amount of pollen in the region where the user 10 exists. Further, in this case, the condition information may include presence/absence of sneezes by the user 10. Further, in this case, the predetermined medicine is, for example, medicine to be taken only when necessary to reduce sneezes by pollen.

Further, in the present embodiment, the user terminal 100 acquires the body condition of the user 10. However, the body condition of the user 10 may not be necessarily acquired by the user terminal 100. The body condition of the user 10 may be acquired, for example, by a camera existing around the user 10. In this case, the camera existing around the user 10 acquires information about presence/absence of coughs or sneezes by the user 10 or the like by picking up images of the user 10. Further, the camera existing around the user 10 may acquire the body condition of the user 10 such as the body temperature, by picking up images of the user 10.

### Second Embodiment

In the first embodiment, the management server 300 executes the predetermined judgment process based on the condition information, the environment information and the history information. Meanwhile, in the present embodiment, the management server 300 executes the predetermined judgment process using information obtained by performing statistical processing of body conditions of a plurality of users including the user 10 and environmental conditions of the users, instead of the history information. Hereinafter, only points different from the first embodiment will be described.

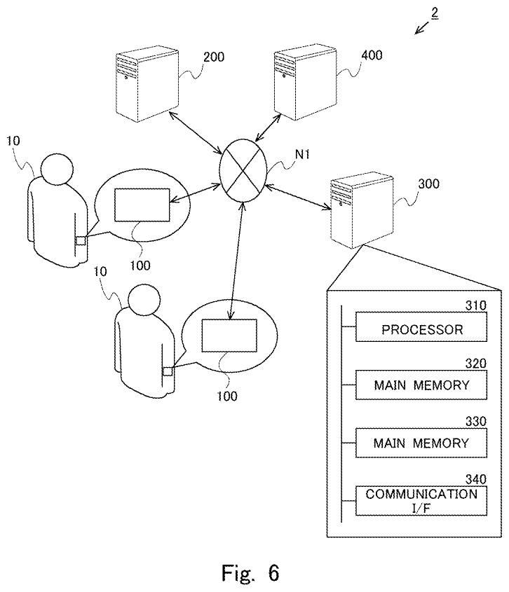

A management system 2 in the present embodiment will be described based on FIG. 6. FIG. 6 is a diagram illustrating a schematic configuration of the management system 2 in the present embodiment. The management system 2 in the present embodiment includes a plurality of user terminals 100 related to the plurality of users 10.

(Functional Configuration)

### (Management Server 300)

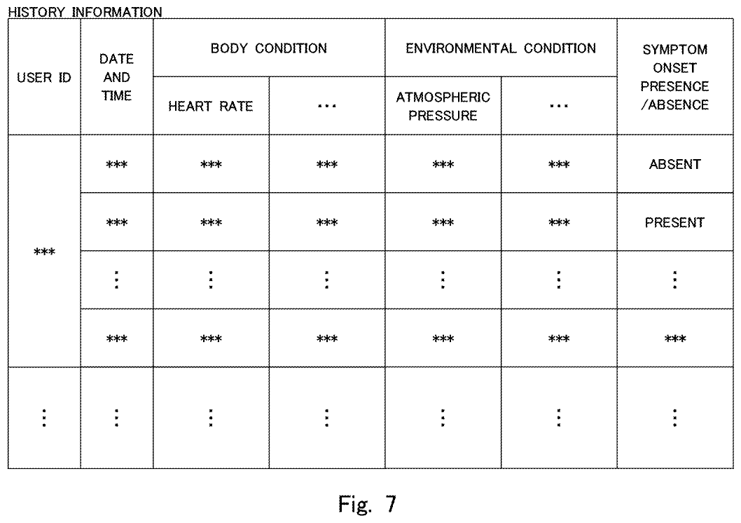

In the history information DB 303, body conditions of the users 10 and environmental conditions of regions where the users 10 existed in a past period are stored. Here, the past period is a period before each user 10 uses predetermined medicine. The length of the past period may be different for each user 10. FIG. 7 is a diagram illustrating an example of a table configuration of the history information in the present embodiment. As illustrated in FIG. 7, the history information includes user ID fields, date and time fields, body condition fields, environmental condition fields and symptom onset presence/absence fields. In the user ID fields, identifiers for identifying the plurality of users 10 are input. In the body condition fields and the environmental condition fields, body conditions about the users 10 and environmental conditions about regions where the users 10 existed are input.

The controller 301 acquires the history information stored in the history information DB 303. By acquiring the history information, the controller 301 acquires statistical information about the body conditions of the plurality of users 10 and the environmental conditions about the regions where the plurality of users 10 existed. Here, the statistical information is acquired, for example, by the controller 301 statistically calculating a correlation between the body conditions of the plurality of users 10 and the environmental conditions of the regions where the plurality of the users 10 existed in the history information. Then, the controller 301 executes a predetermined judgment process for judging whether predetermined symptoms occur on user 10 based on condition information about the user 10, environmental information about the user 10 and the statistical information. Specifically, the controller 301 judges whether the body condition about the user 10, the environmental condition about the user 10 and the statistical information satisfy a predetermined condition or not. Here, the predetermined condition is that a body condition in the condition information and an environmental condition in the environmental information are similar to a body condition and an environmental condition in the statistical information.

(Notification Process)

Since a notification process executed by the controller 301 of the management server 300 in the management system 2 according to the present embodiment is similar to that of the first embodiment, description thereof will be omitted.

As described above, it is also possible to prompt the user 10 to use the predetermined medicine that is irregularly used, at an appropriate timing by the management system 2 of the present embodiment.

### Other Embodiments

The embodiments stated above are mere examples, and the present disclosure can be appropriately changed and implemented within a range not departing from its spirit. Further, the processes and structures described in the present disclosure can be freely combined and implemented as far as technical contradiction does not occur.

Further, a process described as being performed by one apparatus may be shared and executed by a plurality of apparatuses. Alternatively, processes described as being performed by different apparatuses may be executed by one apparatus. In a computer system, by what hardware configuration (server configuration) each function is realized can be flexibly changed.

The present disclosure can be realized by supplying a computer program in which the functions described in the above embodiments are implemented to a computer, and one or more processors that the computer has reading out and executing the program. Such a computer program may be provided to the computer by a non-transitory computer-readable storage medium connectable to a system bus of the computer or may be provided to the computer via a network. The non-transitory computer-readable storage medium includes, for example, a disk of an arbitrary type such as a magnetic disk (a floppy (registered trademark) disk, a hard disk drive (HDD) or the like), an optical disk (a CD-ROM, a DVD disc, a Blu-ray disc or the like), and a medium of an arbitrary type that is appropriate for storing electronic instructions like a read-only memory (ROM), a random access memory (RAM), an EPROM, an EEPROM, a magnetic card, a flash memory or an optical card.

What is claimed is:

1. An information processing apparatus comprising a controller comprising at least one processor configured to execute:
acquiring first information about a condition of a body of a user;
acquiring second information about a condition of an environment around the user;
executing a predetermined judgment process for judging whether predetermined symptoms occur on the user within a predetermined period, based on the first information and the second information; and
notifying the user that predetermined medicine for coping with the predetermined symptoms is to be used, if a positive judgment is made in the predetermined judgment process.

2. The information processing apparatus according to claim 1, wherein
the controller further executes acquiring history information about the condition of the body of the user and the condition of the environment around the user, and executes the predetermined judgment process further based on the history information.

3. The information processing apparatus according to claim 1, wherein
the controller further executes acquiring statistical information about conditions of bodies of a plurality of users including the user and conditions of environments around the plurality of users, and executes the predetermined judgment process further based on the statistical information.

4. The information processing apparatus according to claim 1, wherein
the controller further executes:
estimating symptom onset time when the predetermined symptoms occur on the user, if a positive judgment is made in the predetermined judgment process;
identifying use time for the user to use the predetermined medicine, based on the symptom onset time; and
notifying the user of the use time.

5. The information processing apparatus according to claim 4, wherein
the controller further executes acquiring time when the user used the predetermined medicine last, and identifies the use time based on the time when the user used the predetermined medicine last.

6. The information processing apparatus according to claim 1, wherein
the controller further executes transmitting, after notifying the user that the predetermined medicine is to be used, information about conditions of the body of the user and conditions of the environment around the user in periods before and after a time point of the user using the predetermined medicine, to a server apparatus related to a medical institution.

7. The information processing apparatus according to claim 1, wherein
the second information includes information about forecast change in the condition of the environment around the user.

8. An information processing method comprising:
acquiring first information about a condition of a body of a user;
acquiring second information about a condition of an environment around the user;
executing a predetermined judgment process for judging whether predetermined symptoms occur on the user within a predetermined period, based on the first information and the second information; and
notifying the user that predetermined medicine for coping with the predetermined symptoms is to be used, if a positive judgment is made in the predetermined judgment process.

9. The information processing method according to claim 8, further comprising acquiring history information about the condition of the body of the user and the condition of the environment around the user; wherein
the predetermined judgment process is executed further based on the history information.

10. The information processing method according to claim 8, further comprising acquiring statistical information about conditions of bodies of a plurality of users including the user and conditions of environments around the plurality of users; wherein
the predetermined judgment process is executed further based on the statistical information.

11. The information processing method according to claim 8, further comprising:
estimating symptom onset time when the predetermined symptoms occur on the user, if a positive judgment is made in the predetermined judgment process;
identifying use time for the user to use the predetermined medicine, based on the symptom onset time; and
notifying the user of the use time.

12. The information processing method according to claim 11, further comprising acquiring time when the user used the predetermined medicine last, wherein
the use time is identified based on the time when the user used the predetermined medicine last.

13. The information processing method according to claim 8, further comprising transmitting, after notifying the user that the predetermined medicine is to be used, information about conditions of the body of the user and conditions of the environment around the user in periods before and after a time point of the user using the predetermined medicine, to a server apparatus related to a medical institution.

14. The information processing method according to claim 8, wherein
the second information includes information about forecast change in the condition of the environment around the user.

15. A non-transitory storage medium storing a program causing a computer to execute an information processing method, the information processing method comprising:
acquiring first information about a condition of a body of a user;
acquiring second information about a condition of an environment around the user;
executing a predetermined judgment process for judging whether predetermined symptoms occur on the user within a predetermined period, based on the first information and the second information; and
notifying the user that predetermined medicine for coping with the predetermined symptoms is to be used, if a positive judgment is made in the predetermined judgment process.

16. The non-transitory storage medium according to claim 15, wherein
the information processing method further comprises acquiring history information about the condition of the body of the user and the condition of the environment around the user; and
the predetermined judgment process is executed further based on the history information.

17. The non-transitory storage medium according to claim 15, wherein
the information processing method further comprises acquiring statistical information about conditions of bodies of a plurality of users including the user and conditions of environments around the plurality of users; and
the predetermined judgment process is executed further based on the statistical information.

18. The non-transitory storage medium according to claim 15, wherein
the information processing method further comprises:
estimating symptom onset time when the predetermined symptoms occur on the user, if a positive judgment is made in the predetermined judgment process;
identifying use time for the user to use the predetermined medicine, based on the symptom onset time; and
notifying the user of the use time.

19. The non-transitory storage medium according to claim 18, wherein
the information processing method further comprises acquiring time when the user used the predetermined medicine last; and
the use time is identified based on the time when the user used the predetermined medicine last.

20. The non-transitory storage medium according to claim 15, wherein
the information processing method further comprises transmitting, after notifying the user that the predetermined medicine is to be used, information about conditions of the body of the user and conditions of the environment around the user in periods before and after a time point of the user using the predetermined medicine, to a server apparatus related to a medical institution.
